(19) United States
(12) Patent Application Publication
(10) Pub. No.: US 2008/0020421 A1
(43) Pub. Date: Jan. 24, 2008

(54) METHODS OF SCREENING AGENTS FOR CYTOTOXIC AND ANTIMICROBIAL ACTIVITY

(76) Inventor: Renato J. Aguilera, El Paso, TX (US)

Correspondence Address: FULBRIGHT & JAWORSKI L.L.P., 600 CONGRESS AVE., SUITE 2400, AUSTIN, TX, 78701, US

(21) Appl. No.: 11/780,291

(22) Filed: Jul. 19, 2007

Related U.S. Application Data
(60) Provisional application No. 60/807,963, filed on Jul. 21, 2006.

(51) Int. Cl. C12Q 1/02

(52) U.S. Cl. 435/32

(57) ABSTRACT

The present invention provides for methods of screening agents for cytotoxic and/or anti-microbial activity. In particular embodiments, the loss of a fluorescent signal from the treated cells, due to cell death or inhibition of growth, is measured for several different cell types in parallel.

This application claims benefit of priority to U.S. Provisional Application Ser. No. 60/807,963, filed Jul. 21, 2006, the entire contents of which are hereby incorporated by reference.

This invention was made with government support under grant numbers 2G12RR08124, S06 GM8012-34 and AI01812-02 awarded by the National Institutes of Health. The government has certain rights in the invention.

### BACKGROUND OF THE INVENTION

1. Field of the Invention

The present invention relates generally to the fields of cell biology, molecular biology, oncology and microbiology. More particularly, it concerns the use of novel screening assays for cytotoxic and anti-microbial agents.

2. Description of Related Art

As large quantities of novel chemical compounds are generated, companies and researchers are faced with the growing challenge of screening these compounds for their potential therapeutic and/or toxic effects; thus leading to a growing demand for novel cell-based high-throughput screening assays (Kassel, 2004; Kenseth and Coldiron, 2004; Zemanova et al., 2003). A significant proportion of potential anticancer drugs exhibit unacceptable levels of toxicity against normal cells and tissues and for this reason it is imperative to pre-screen such compounds in vitro (Frankfurt and Krishan, 2003a; Frankfurt and Krishan, 2003b). The inventors have recently reported a relatively simple fluorescence-based toxicity screening assay (Montoya et al., 2004) that involves the detection of DNA degradation in a human cancer cell line that constitutively expresses GFP in the nuclei (Kanda et al., 1998). Due to the stable nuclear localization of the histone H2B-GFP-fusion protein, altered apoptotic and/or necrotic nuclei can be readily detected in situ by fluorescence microscopy. Several structurally similar naphthoquinone compounds, including the well known anti-cancer compound plumbagin, were screened using this assay (Montoya et al., 2004). From this initial screen of eleven compounds, two novel naphthoquinones were found to be more toxic than plumbagin. Subsequent assays revealed that the most toxic naphthoquinones, including plumbagin, elicited both apoptotic and necrotic modes of cell death. These results were consistent with the generation of reactive oxygen species (ROS) as demonstrated with plumbagin (Inbaraj and Chignell, 2004). In a related analysis, the HeLa-GFP assay was used to evaluate the potential toxicity of a small library of acetophenone compounds with potential antimicrobial properties (Rajabi et al., 2005). Although some of the antibacterial acetophenone compounds exhibited varying levels of cytotoxicity, three of the most promising compounds were determined to exert their activity selectively against M. avium (Rajabi et al., 2005). Unfortunately, the HeLa-GFP assay the inventors previously reported could not be applied to high-throughput analyses as it relied on the visual detection of cell death through fluorescence microscopy. For this reason, the inventors improved their assays to incorporate high content screening using microtiter plates (Montoya et al., 2005).

Although mycobacterial infections are on the increase worldwide, particularly in immunocompromised populations as is the case of M. avium in AIDS patients, there have been no new drugs developed against these organisms since the 1960's (Rajabi et al., 2005, and references within). The emergence of drug-resistant bacteria has also lead to an increased interest in the generation of novel antibiotics. Thus, new and improved methods of screening for such agents are urgently needed.

### SUMMARY OF THE INVENTION

Thus, in accordance with the present invention, there is provided a method of screening a test substance for cytotoxic and antimicrobial activity comprising (a) providing at least one eukaryotic cell and at least one pathogenic organism, each of the cells expressing a fluorescent marker protein or stained with a fluorescent dye; (b) contacting each of the cells with the test substance; and (c) assessing a fluorescent signal from each of the cell or organism, wherein a decrease in signal from only the eukaryotic cell indicates that the test substance is a cytotoxic agent, and wherein a decrease in signal from only the pathogenic organism indicates that the test substance is an antimicrobial agent, and wherein a decrease in signal from both the eukaryotic cell and pathogenic organism indicates that the test substance is both a cytotoxic agent and an antimicrobial agent. The test substance may be a protein, a peptide, a nucleic acid, an organopharmaceutical, a lipid, or a carbohydrate. The method may further comprise more than one concentration of the test substance against each of the cells, and/or may further comprise testing multiple replicates of each cell and concentration tested.

The eukaryotic cell may be a cancer cell, such as a multi-drug resistant cancer cell. The pathogenic organism may be a prokaryotic organism, such as a bacterium or a protozoan, such as a multi-drug resistant bacterial cell. The pathogenic organism could also be a yeast or fungus. The fluorescent marker protein may be GFP, BFP, CFP, YFP, EGFP, EYFP, Venus, Citrine, phiYFP, copGFP CGFP, ECFP, Cerulean, CyPet, T-Sapphire, Emerald, YPet, AcGFP1, AmCyan, AsRed2, dsRed, dsRed2, dsRed-Express, EBFP, HcRed, ZsGreen, ZsYellow, J-Red, TurboGFP, Kusabira Orange, Midoriishi Cyan, mOrange, DsRed-monomer, mStrawberry, mRFP1, tdTomato, mCherry, mPlum, and mRaspberry. The fluorescent dye may be CellTracker Green™. The fluorescent marker protein or dye in the eukaryotic cell and pathogenic organism may be the same or different.

The step of providing may comprise distributing the eukaryotic cell into at least one well of a first multi-well plate, and distributing the pathogenic organism into at least one well of a second multi-well plate. The multi-well plate may be a 6-well, 24-well, 96-well or high throughput plate. Assessing may comprise fluorescence detection via commercially available devices. The method may further comprise assessing the fluorescent signal from a control eukaryotic cell and control prokaryotic cell that each is detectable with a fluorescent marker protein or dye as the cells in step (a), wherein the control cells have not been treated with the test substance.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

The use of the term “or” in the claims is used to mean “and/or” unless explicitly indicated to refer to alternatives only or the alternative are mutually exclusive, although the disclosure supports a definition that refers to only alternatives and “and/or.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—Chemical structure of the naphthoquinone compounds that were synthesized for this study (see Examples for additional details).

FIG. 2—Determination of the lethal dose (LD) of cytotoxic naphthoquinone compounds. Several of the most toxic compounds detected with initial screening assay were re-tested at various concentrations to determine the LD50 in HeLa-GFP. The concentration of the test compounds (μM) are shown within the figure. Horizontal bar indicates the 50% toxicity range. H₂O₂ (+control, 1 mM) was used as a positive control (+; 100% cytolysis) while cells treated with DMSO (1 μl; solvent for all quinones) was used as the negative (−) control.

Table 1—Percent GFP-fluorescence after incubation with test compounds.

### DESCRIPTION OF ILLUSTRATIVE EMBODIMENTS

### I. The Present Invention

A fluorescence-based assay has been developed for the rapid screening of potential cytotoxic and bacteriocidal compounds. The assay is exemplified using detection of GFP (Green Fluorescent Protein) in HeLa human carcinoma cells as well as gram-negative (Echerichia coli) and gram-positive bacteria (Mycobacterium avium). Addition of a toxic compound to the GFP marked cells resulted in the loss of the GFP fluorescence which was readily detected by fluorometry. Thirty-nine distinct naphthoquinone-derivatives were screened and several of these compounds were found to be toxic to all cell types. Apart from differences in overall toxicity, two general types of toxic compounds were detected, those that exhibited toxicity to two or all three of the cell types and those that were primarily toxic to the HeLa cells. These results demonstrate that the parallel screening of both eukaryotic cells and pathogenic organisms is not only feasible and reproducible but also cost effective. The exploitation of this assay is described in detail in the following pages.

### II. Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny. As used herein, the terms “engineered” and “recombinant” cells or host cells are intended to refer to a cell into which an exogenous nucleic acid sequence, such as, for example, a vector, has been introduced. Therefore, recombinant cells are distinguishable from naturally-occurring cells which do not contain a recombinantly introduced nucleic acid.

In certain embodiments, it is contemplated that RNAs or proteinaceous sequences may be co-expressed with other selected RNAs or proteinaceous sequences in the same host cell. Co-expression may be achieved by co-transfecting the host cell with two or more distinct recombinant vectors. Alternatively, a single recombinant vector may be constructed to include multiple distinct coding regions for RNAs, which could then be expressed in host cells transfected with the single vector.

In certain embodiments, the host cell may be a pathogenic organism. In certain embodiments, the organism may be, but is not limited to, a prokaryote (e.g., a eubacteria, an archaea) or an eukaryote, as would be understood by one of ordinary skill in the art (see, for example, webpage phylogeny.arizona.edu/tree/phylogeny.html). Pathogens include bacteria, yeast, fungi, or higher eukaryotes such as parasites like trypanosomes. Examples of fungal cells are filamentous bacteria, for example Aspergillus, Neurospora, and Fusarium, and cells belonging to Trichoderma. When a filamentous bacterium is used as a host cell, transformation can be performed by integrating the DNA construct into the host chromosome and obtaining a recombinant host cell. Integration of the DNA construct into the host chromosome can be performed according to a known method such as homologous recombination or heterologous recombination.

1. Eukaryotic Cells

Examples of eukaryotic host cells for replication and/or expression of a vector include, but are not limited to cancer or transformed cell lines such as HeLa, NIH3T3, Jurkat, 293, Cos, CHO, Saos, and PC12. Many host cells from various cell types and organisms are available and would be known to one of skill in the art. Examples of yeast cells include cells belonging to Saccharomyces or Schizosaccharomyces, and examples include Saccharomyces cerevisae and Saccharomyces kluyveri.

When an insect cell is used as a host, a protein can be expressed by co-transfecting a recombinant gene transduction vector and a baculovirus into the insect cell to obtain a recombinant virus in the insect cell culture supernatant, and then infecting the recombinant virus into the insect cell (for example, as described in Baculovirus Expression Vectors, 1988, and the like).

As a baculovirus, for example, Autographa californica nuclear polyhedrosis virus, which is a virus that infects Mamestra-family insects, and the like, can be used. As an insect cell, for example, Sf9 and Sf21, which are ovarian cells of Spodoptera frugiperda, and Hi Five, which is an ovarian cell of Trichoplusia ni (Invitrogen), or the like can be used.

2. Prokaryotic Cells

Numerous bacterial cells and cultures are available from the American Type Culture Collection (ATCC), which is an organization that serves as an archive for living cultures and genetic materials (www.atcc.org). Cell types available for vector replication and/or expression include, but are not limited to, bacteria such as Gram-negatives like E. coli (e.g., E. coli strain RR1, E. coli LE392, E. coli B, E. coli X 1776 (ATCC No. 31537) as well as E. coli W3110 (F-, lambda-, prototrophic, ATCC No. 273325), DH5αα, JM109, and KC8, Gram-positive bacteria such as Streptomyces, Bacillus species (e.g., subtilis); and other enterobacteriaceae such as Salmonella typhimurium, Serratia marcescens, various Pseudomonas species, Mycoplasma species (M. avium, M. tuberculosis) as well as a number of commercially available bacterial hosts such as SURE® Competent Cells and SOLOPACK™ Gold Cells (STRATAGENE®, La Jolla).

### III. Screening Assays

Thus, in accordance with the present invention, methods are provided for identifying agents that are cytotoxic and/or have antimicrobial activity. To identify a potential cytotoxic or anti-microbial agent, one will assess the loss of fluorescent signal (either intrinsic through fluorescent protein expression or by fluorescent dye). For example, a method generally comprises:
(a) providing at least one eukaryotic cell and at least one pathogenic organism, each of said cells expressing a fluorescent marker protein or stained with a fluorescent dye;
(b) contacting each of said cells with said test substance; and
(c) assessing a fluorescent signal from each of said cell and organism,
   wherein a decrease in signal from only said eukaryotic cell indicates that the test substance is a cytotoxic agent, and wherein a decrease in signal from only said pathogenic organism indicates that the test substance is an antimicrobial agent, and wherein a decrease in signal from both said eukaryotic cell and said pathogenic organism indicates that the test substance is both a cytotoxic agent and an antimicrobial agent.

As used herein the term “candidate substance” refers to any molecule that may exhibit the desired activity. The candidate substance may be any molecule including a protein or fragment thereof, a small molecule, or even a nucleic acid molecule. These can be natural compounds, synthetic compounds or synthetic modifications of natural compounds. “Rational drug design” includes not only comparisons with known inhibitors and activators, but predictions relating to the structure of target molecules. The goal of rational drug design is to produce structural analogs of biologically active polypeptides or target compounds. By creating such analogs, it is possible to fashion drugs, which are more active or stable than the natural molecules, which have different susceptibility to alteration or which may affect the function of various other molecules. In one approach, one would generate a three-dimensional structure for a target molecule, or a fragment thereof. This could be accomplished by x-ray crystallography, computer modeling or by a combination of both approaches.

One may simply acquire, from various commercial or non-commercial sources, small molecule libraries that are believed to meet the basic criteria for useful drugs in an effort to “brute force” the identification of useful compounds. Screening of such libraries, including combinatorially generated libraries, is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecules or any other compounds that may be designed through rational drug design starting from known inhibitors or stimulators.

### IV. Fluorescent Dyes

A variety of dyes may be employed according to the present invention, including many that are commercially available. The following dyes are available from Molecular Probes division of Invitrogen:
calcein blue, AM
calcein, AM
calcein, AM *1 mg/mL solution in anhydrous DMSO*
calcein, AM—special packaging
CellTrace™ BODIPY® TR methyl ester *lipophilic counterstain for GFP* *solution in DMSO*
CellTrace™ calcein blue, AM—special packaging
CellTrace™ calcein green, AM—special packaging
CellTrace™ calcein red-orange, AM—special packaging
CellTrace™ calcein violet, AM *for 405 nm excitation* *special packaging*
CellTrace™ Far Red DDAO-SE—special packaging
CellTrace™ Oregon Green® 488 carboxylic acid diacetate, succinimidyl ester (carboxy-DFFDA, SE) *cell permeant* *mixed isomers*
CellTracker™ Blue CMAC (7-amino-4-chloromethylcoumarin)
CellTracker™ Blue CMF2HC (4-chloromethyl-6,8-difluoro-7-hydroxycoumarin)
CellTracker™ Blue CMHC (4-chloromethyl-7-hydroxycoumarin)
CellTracker™ Green BODIPY® (8-chloromethyl-4,4-difluoro-1,3,5,7-tetramethyl-4-bora-3a,4a-diaza-s-indacene)
CellTracker™ Green CMFDA (5-chloromethylfluorescein diacetate)—special packaging
CellTracker™ Green CMFDA (5-chloromethylfluorescein diacetate), 1 mg
CellTracker™ Orange CMRA—special packaging
CellTracker™ Orange CMTMR (5-(and-6)-(((4-chloromethyl)benzoyl)amino)tetramethylrhodamine)—mixed isomers
CellTracker™ Red CMTPX—special packaging
   Biotium, a leading fluorescence technology company is offering six fluorescent phalloidin products for F-actin detection. Each fluorescent phalloidin has its own unique features.

Rhodamine 110 Phalloidin (Cat: 00032):
green fluorescent (λₑₓ/λₑₘ=496/520 nm)
more photostable than FITC and Alexa 488
detected with popular optical filter sets for FITC
economical: only 66 cents per assay

Fluorescein Phalloidin (Cat: 00030):
green fluorescent (λₑₓ/λₑₘ=494/518 nm)
economical: only 66 cents per assay

Blue 405 Phalloidin (Cat: 00034):
blue fluorescent (λₑₓ/λₑₘ=405/460 nm)
detected with popular optical filter sets for DAPI
economical: only 66 cents per assay

Rhodamine Phalloidin (Cat: 00027):
red fluorescent (λₑₓ/λₑₘ=542/565 nm)
economical: only 66 cents per assay

Sulforhodamine 101 (Texas-Red) Phalloidin (Cat: 00033):
red fluorescent (λₑₓ/λₑₘ=593/613 mm)
economical: only 66 cents per assay

Biotin-XX Phalloidin (Cat#00028):
long flexible spacer XX facilitates binding to avidin or streptavidin
versatile: easily linking to fluorophore, enzyme for fluorescence or colorimetric visualization of F-actin
can be used for detecting F-actin by electron microscopy
Economical: $2.5 per assay
   Biotium also provides over 30 bioassay kits and 500 fluorescent and bioluminescent products for bioscience research and drug screening (www.biotium.com).

### V. Cells Engineered with Fluorescent Molecules

In another embodiment, the present invention involves the use of cells that have been engineered to express a screenable marker protein, e.g., a fluorescent protein. The marker is regulated by an appropriate promoter active in the host cell. The following provides a discuss of relevant technologies for this embodiment.

1. Fluorescent Proteins

Aequorea victoria are brightly luminescent jellyfish, with glowing points around the margin of the umbrella. Light arises from yellow tissue masses that each consist of about 6000-7000 photogenic cells. The cytoplasm of these cells is densely packed with fine granules that contain a Ca++ activated photoprotein, aequorin, that emits blue-green light, and an accessory green fluorescent protein (GFP), which accepts energy from aequorin and re-emits it as green light. GFP is an extremely stable protein of 238 amino acids, stable in neutral buffers up to 65° C., and displaying a broad range of pH stability from 5.5 to 12. The protein is intensely fluorescent, with a quantum efficiency of approximately 80% and molar extinction coefficient of 2.2×10⁴ cm−1 M−1 (after correction for the known molecular weight). GFP fluoresces maximally when excited at 400 nm with a lesser peak at 475 nm, and fluorescence emission peaks at 509 nm. Since the purification and cloning of the GFP from Aequorea Victoria, similar fluorescent proteins have been isolated and cloned from many other species including dynoflagelates, sea pens, and reef corals.

Two variants of the Aequorea Victoria GFP, cyan fluorescent protein (CFP) and yellow fluorescent protein (YFP), have special fluorescence excitation and emission properties that are well suited to measurement of close molecular distances. Thus, variants of this type have been the most widely used for FRET experiments. When these two molecules are positioned at distances closer than 7 nm of each other, energy transfer can occur from the excited state of the donor molecule (CFP), to the unoccupied excited state of the acceptor molecule (YFP) by a process commonly referred to as fluorescence (or Förster) resonance energy transfer (FRET). FRET between CFP and YFP can be detected using a wide variety of spectroscopic and fluorescence microscopy techniques and is often used to report protein-protein interactions or changes in the conformation state of a peptide or protein. Since the efficiency of FRET is directly related to the spectroscopic properties of both the donor and acceptor molecules, improvements can be made to the fluorescence properties of the fluorophores, such as to increase both the FRET efficiency and the chances of successful detection. FRET has also been used to quantify association of a protein of interest with an organelle (Chiu et al., 2002). As more fluorescent proteins are developed, other FRET pairs are being tried, such as the CFP to an Orange FP from reef coral, called mKO (Karasawa et al., 2004). One of the strengths of the present invention is that it is broadly applicable to any pair of fluorescent proteins that have spectral properties sufficient to generate FRET.

The gene of the green fluorescent protein has been isolated and its sequence has also been determined (Prasher et al., 1992). There have also been numerous reports of amino acid sequences of other fluorescent proteins or their mutants, for example, as described in Tsien (1998) and the literature cited therein. Fluorescent proteins include green fluorescent protein, red fluorescent protein, yellow fluorescent protein, GFP, BFP, CFP, YFP, EGFP, EYFP, Venus, Citrine, phiYFP, copGFP CGFP, ECFP, Cerulean, CyPet, T-Sapphire, Emerald, YPet, AcGFP1, AmCyan, AsRed2, dsRed, dsRed2, dsRed-Express, EBFP, HcRed, ZsGreen, ZsYellow, J-Red, TurboGFP, Kusabira Orange, Midoriishi Cyan, mOrange, DsRed-monomer, mStrawberry, mRFP1, tdTomato, mCherry, mPlum, and mRaspberry.

The term “cyan fluorescent protein (CFP)” as used herein is defined as any fluorescent protein with an absorption maximum between 420 and 460 nm, and a fluorescence maximum between 460 and 500 nm. These proteins have mainly been derived from the wild-type Aequoria GFP with a Y66W mutation, resulting in a primary excitation peak at ˜434 nm with minor excitation maxima at ˜452 nm, and a primary emission peak is 477 nm with minor shoulder at ˜505 nm (Heim et al., 1994). Other fluorescent proteins are termed “green fluorescent protein (GFP)”, meaning proteins with absorption maxima between 480 and 500 nm and fluorescence maxima between 500 and 515 nm, and “yellow fluorescent protein (YFP)” meaning proteins with absorption maxima between 500 and 520 nm and fluorescence maxima between 515 and 535 nm.

2. Cell Engineering Materials and Methods

Embodiments of the present invention concern a nucleic acid encoding fluorescent proteins. The term “nucleic acid” is well known in the art, and generally refers to a molecule (i.e., a strand) of DNA, RNA or a derivative or analog thereof. The term “nucleic acid” encompasses the terms “oligonucleotide” and “polynucleotide,” each as a subgenus of the term “nucleic acid.” The term “oligonucleotide” refers to a molecule of between about 3 to about 50 nucleobases in length. The term “polynucleotide” refers to at least one molecule of greater than about 50 nucleotides in length.

A nucleic acid may be made by any technique known to one of ordinary skill in the art, such as for example, chemical synthesis, enzymatic production or biological production. Non-limiting examples of a synthetic nucleic acid (e.g., a synthetic oligonucleotide), include a nucleic acid made by in vitro chemically synthesis using phosphotriester, phosphite or phosphoramidite chemistry and solid phase techniques such as described in EP 0 266 032, incorporated herein by reference, or via deoxynucleoside H-phosphonate intermediates as described by Froehler et al. (1986) and U.S. Pat. No. 5,705,629, each incorporated herein by reference. In the methods of the present invention, one or more oligonucleotide may be used. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid include one produced by enzymes in amplification reactions such as PCR™ (see for example, U.S. Pat. No. 4,683,202 and U.S. Pat. No. 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al., 2001, incorporated herein by reference).

In certain aspect, the present invention concerns a nucleic acid that is an isolated nucleic acid. As used herein, the term “isolated nucleic acid” refers to a nucleic acid molecule (e.g., an RNA or DNA molecule) that has been partially or substantially isolated free of total genomic and in vitro reaction products. In certain embodiments, “isolated nucleic acid” also refers to a nucleic acid that has been isolated free the bulk of cellular components or in vitro reaction components. A nucleic acid may be purified on polyacrylamide gels, cesium chloride centrifugation gradients, or by any other means known to one of ordinary skill in the art (see for example, Sambrook et al., 2001, incorporated herein by reference).

a. Vectors

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Maniatis et al., 1990 and Ausubel et al., 1996, both incorporated herein by reference).

The term “expression vector” refers to any type of genetic construct comprising a nucleic acid coding for a RNA capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well and are described infra. A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30-110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the β-lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression (see, for example Sambrook et al. 2001, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally any promoter/enhancer combination (as per, for example, the Eukaryotic Promoter Data Base EPDB, www.epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Examples of promoters which are operative in bacterial cells include, a promoter of Bacillus stearothermophilus maltogenic amylase gene, Bacillus licheniformis α-amylase gene, Bacillus amyloliquefaciens BAN amylase gene, Bacillus subtilis alkaline protease gene, or Bacillus pumilus xylosldase gene; a P_{R} or P_{L} promoter of phage lambda; a lac, trp, or tac promoter of Escherichia coli; and the like.

Examples of promoters which are operative in insect cells include polyhedrin promoter, P10 promoter, basic protein promoter of Autographa californica nuclear polyhedrosis, baculovirus immediate early gene 1 promoter, baculovirus 39K delayed early gene promoter, and the like. Examples of promoters which are operative in yeast host cells include a promoter derived from yeast glycolysis system genes, alcohol dehydrogenase gene promoter, TPI1 promoter, ADH2-4-c promoter, and the like.

In certain embodiments, a plasmid vector is contemplated for use to transform a host cell. In general, plasmid vectors containing replicon and control sequences which are derived from species compatible with the host cell are used in connection with these hosts. The vector ordinarily carries a replication site, as well as marking sequences which are capable of providing phenotypic selection in transformed cells. In a non-limiting example, E. coli is often transformed using derivatives of pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, for example, promoters which can be used by the microbial organism for expression of its own proteins.

In addition, phage vectors containing replicon and control sequences that are compatible with the host microorganism can be used as transforming vectors in connection with these hosts. For example, the phage lambda GEM™-11 may be utilized in making a recombinant phage vector which can be used to transform host cells, such as, for example, E. coli LE392. Further useful plasmid vectors include pIN vectors (Inouye et al., 1985), and pGEX vectors, for use in generating glutathione S-transferase (GST) soluble fusion proteins for later purification and separation or cleavage. Other suitable fusion proteins are those with β-galactosidase, ubiquitin, and the like.

Another vector of interest is the “PEST” vector, pd4EGFP-Bid, from Clontech. This is a mammalian expression vector that encodes a fusion of destabilized enhanced green fluorescent protein (d4EGFP) and Bid, a member of the Bcl-2 “pro-apoptosis” family. Because of its fluorescent label, the Bid-d4EGFP fusion is easily detected by microscopy, allowing researchers to track its movements in response to certain apoptotic stimuli. To drive expression of the fluorescent fusion, pd4EGFP-Bid contains the immediate early promoter of cytomegalovirus, positioned just upstream of the Bid sequence. A short linker joins the Bid coding sequence to the 5′-end of d4EGFP. Farther downstream, the vector contains a pair of SV40 polyadenylation signals, which direct proper processing of the 3′ end of the Bid-d4EGFP mRNA. The vector also contains an SV40 origin for replication in mammalian cells expressing the SV40 T antigen, a pUC origin of replication for propagation in E. coli, and an f1 origin for single-stranded DNA production. A neomycin-resistance cassette (Neo^{r}), consisting of the SV40 early promoter, the neomycin/kanamycin resistance gene of Tn5, and polyadenylation signals from the HSV TK gene, allows stably transfected eukaryotic cells to be selected using G418. A bacterial promoter upstream of the cassette confers kanamycin resistance (Kan^{r}) to E. coli. d4EGFP, a destabilized, red-shifted excitation variant of Aequorea victoria GFP, serves not only as a label, but also as a device to ensure protein turnover.

Bacterial host cells, for example, E. coli, comprising the expression vector, are grown in any of a number of suitable media, for example, LB. The expression of the recombinant protein in certain vectors may be induced, as would be understood by those of skill in the art, by contacting a host cell with an agent specific for certain promoters, e.g., by adding IPTG to the media or by switching incubation to a higher temperature. After culturing the bacteria for a further period, generally of between 2 and 24 h, the cells are collected by centrifugation and washed to remove residual media.

b. Vector Delivery and Cell Transformation

Suitable methods for nucleic acid delivery for transformation of an organelle, a cell, a tissue or an organism for use with the current invention are believed to include virtually any method by which a nucleic acid (e.g., DNA) can be introduced into an organelle, a cell, a tissue or an organism, as described herein or as would be known to one of ordinary skill in the art. Such methods include, but are not limited to, direct delivery of DNA such as by ex vivo transfection (Wilson et al., 1989, Nabel et al., 1989), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985), and any combination of such methods. Through the application of techniques such as these, organelle(s), cell(s), tissue(s) or organism(s) may be stably or transiently transformed.

i. Injection

In certain embodiments, a nucleic acid may be delivered to an organelle, a cell, a tissue or an organism via one or more injections (i.e., a needle injection), such as, for example, subcutaneously, intradermally, intramuscularly, intervenously, intraperitoneally, etc. Methods of injection of vaccines are well known to those of ordinary skill in the art (e.g., injection of a composition comprising a saline solution). Further embodiments of the present invention include the introduction of a nucleic acid by direct microinjection. Direct microinjection has been used to introduce nucleic acid constructs into Xenopus oocytes (Harland and Weintraub, 1985).

ii. Electroporation

In certain embodiments of the present invention, a nucleic acid is introduced into an organelle, a cell, a tissue or an organism via electroporation. Electroporation involves the exposure of a suspension of cells and DNA to a high-voltage electric discharge. In some variants of this method, certain cell wall-degrading enzymes, such as pectin-degrading enzymes, are employed to render the target recipient cells more susceptible to transformation by electroporation than untreated cells (U.S. Pat. No. 5,384,253, incorporated herein by reference). Alternatively, recipient cells can be made more susceptible to transformation by mechanical wounding.

Transfection of eukaryotic cells using electroporation has been quite successful. Mouse pre-B lymphocytes have been transfected with human kappa-immunoglobulin genes (Potter et al., 1984), and rat hepatocytes have been transfected with the chloramphenicol acetyltransferase gene (Tur-Kaspa et al., 1986) in this manner.

To effect transformation by electroporation in cells such as, for example, plant cells, one may employ either friable tissues, such as a suspension culture of cells or embryogenic callus or alternatively one may transform immature embryos or other organized tissue directly. In this technique, one would partially degrade the cell walls of the chosen cells by exposing them to pectin-degrading enzymes (pectolyases) or mechanically wounding in a controlled manner. Examples of some species which have been transformed by electroporation of intact cells include maize (U.S. Pat. No. 5,384,253; Rhodes et al., 1995; D'Halluin et al., 1992), wheat (Zhou et al., 1993), tomato (Hou and Lin, 1996), soybean (Christou et al., 1987) and tobacco (Lee et al., 1989).

One also may employ protoplasts for electroporation transformation of plant cells (Bates, 1994; Lazzeri, 1995). For example, the generation of transgenic soybean plants by electroporation of cotyledon-derived protoplasts is described by Dhir and Widholm in International Patent Application No. WO 9217598, incorporated herein by reference. Other examples of species for which protoplast transformation has been described include barley (Lazerri, 1995), sorghum (Battraw et al., 1991), maize (Bhattacharjee et al., 1997), wheat (He et al., 1994) and tomato (Tsukada, 1989).

iii. Calcium Phosphate

In other embodiments of the present invention, a nucleic acid is introduced to the cells using calcium phosphate precipitation. Human KB cells have been transfected with adenovirus 5 DNA (Graham and Van Der Eb, 1973) using this technique. Also in this manner, mouse L(A9), mouse C127, CHO, CV-1, BHK, NIH3T3 and HeLa cells were transfected with a neomycin marker gene (Chen and Okayama, 1987), and rat hepatocytes were transfected with a variety of marker genes (Rippe et al., 1990).

iv. DEAE-Dextran

In another embodiment, a nucleic acid is delivered into a cell using DEAE-dextran followed by polyethylene glycol. In this manner, reporter plasmids were introduced into mouse myeloma and erythroleukemia cells (Gopal, 1985).

v. Sonication Loading

Additional embodiments of the present invention include the introduction of a nucleic acid by direct sonic loading. LTK⁻ fibroblasts have been transfected with the thymidine kinase gene by sonication loading (Fechheimer et al., 1987).

vi. Liposome-Mediated Transfection

In a further embodiment of the invention, a nucleic acid may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution.

Current formulations use a DNA condensing agent, such as protamine sulfate, along with “helper lipids” (Reddy et al., 2002) describe a folate-targeted, cationic lipid based transfection complex was developed and found to specifically transfect folate receptor-expressing cells and tumors. These liposomal vectors were comprised of protamine-condensed plasmid DNA, a mixture of cationic and neutral lipids, and a folic acid-cysteine-polyethyleneglycol-phosphatidylethanolamine (FA-Cys-PEG-PE) conjugate. Overall, folate-labeled formulations produced an 8- to 10-fold increase in expression, as compared with the corresponding non-targeted cationic lipid/DNA formulations.

Hong et al. (1997) describe stable complexes of cationic liposomes with plasmid DNA were prepared by (1) including a small amount of polyethyleneglycol-phospholipid conjugate or (2) condensing the DNA with polyamines prior to the formation of liposome-plasmid complexes. These preparations were stable for months at 4° C. and gave reproducible high transfection activity for in vivo gene delivery. In these formulations cholesterol, not dioleoylphosphatidylethanolamine, was the helper lipid effective for sustaining high transfection activity in vivo.

Commercially available lipofection reagents include CellPhect Transfection Kit (Amersham-Pharmacia Biotech), CytoFectene Reagent (Bio-Rad), CLONrectin Reagent (Clontech), Cytofectin (Glen Research), Perfect Lipid™ Transfection Kit (Invitrogen), EuFectin (JBL Scientific), Lipofectamine™ 2000, Lipofectamine Plus™, Lipofectamine™, DMRIE-C Reagent (Life Technologies), ExGen 500 (MBI Fermentas), TransT LT-1 and LT-2 (PanVera), Transfast™ and Trₓ™ Reagents (Promega), SuperFect™ Transfection Reagent (Qiagen), LipoTAXI™ (Stratagene) and Genetransfer HMG-1,-2 Mixture (Wako Chemicals USA).

vii. Receptor Mediated Transfection

Still further, a nucleic acid may be delivered to a target cell via receptor-mediated delivery vehicles. These take advantage of the selective uptake of macromolecules by receptor-mediated endocytosis that will be occurring in a target cell. In view of the cell type-specific distribution of various receptors, this delivery method adds another degree of specificity to the present invention.

Certain receptor-mediated gene targeting vehicles comprise a cell receptor-specific ligand and a nucleic acid-binding agent. Others comprise a cell receptor-specific ligand to which the nucleic acid to be delivered has been operatively attached. Several ligands have been used for receptor-mediated gene transfer (Wu and Wu, 1987; Wagner et al., 1990; Perales et al., 1994; Myers, EPO 0273085), which establishes the operability of the technique. Specific delivery in the context of another mammalian cell type has been described (Wu and Wu, 1993; incorporated herein by reference). In certain aspects of the present invention, a ligand will be chosen to correspond to a receptor specifically expressed on the target cell population.

In other embodiments, a nucleic acid delivery vehicle component of a cell-specific nucleic acid targeting vehicle may comprise a specific binding ligand in combination with a liposome. The nucleic acid(s) to be delivered are housed within the liposome and the specific binding ligand is functionally incorporated into the liposome membrane. The liposome will thus specifically bind to the receptor(s) of a target cell and deliver the contents to a cell. Such systems have been shown to be functional using systems in which, for example, epidermal growth factor (EGF) is used in the receptor-mediated delivery of a nucleic acid to cells that exhibit upregulation of the EGF receptor.

In still further embodiments, the nucleic acid delivery vehicle component of a targeted delivery vehicle may be a liposome itself, which will preferably comprise one or more lipids or glycoproteins that direct cell-specific binding. For example, lactosyl-ceramide, a galactose-terminal asialganglioside, have been incorporated into liposomes and observed an increase in the uptake of the insulin gene by hepatocytes (Nicolau et al., 1987). It is contemplated that the tissue-specific transforming constructs of the present invention can be specifically delivered into a target cell in a similar manner.

viii. Microprojectile Bombardment

Microprojectile bombardment techniques can be used to introduce a nucleic acid into at least one, organelle, cell, tissue or organism (U.S. Pat. No. 5,550,318; U.S. Pat. No. 5,538,880; U.S. Pat. No. 5,610,042; and PCT Application WO 94/09699; each of which is incorporated herein by reference). This method depends on the ability to accelerate DNA-coated microprojectiles to a high velocity allowing them to pierce cell membranes and enter cells without killing them (Klein et al., 1987). There are a wide variety of microprojectile bombardment techniques known in the art, many of which are applicable to the invention.

In this microprojectile bombardment, one or more particles may be coated with at least one nucleic acid and delivered into cells by a propelling force. Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold particles or beads. Exemplary particles include those comprised of tungsten, platinum, and preferably, gold. It is contemplated that in some instances DNA precipitation onto metal particles would not be necessary for DNA delivery to a recipient cell using microprojectile bombardment. However, it is contemplated that particles may contain DNA rather than be coated with DNA. DNA-coated particles may increase the level of DNA delivery via particle bombardment but are not, in and of themselves, necessary.

For the bombardment, cells in suspension are concentrated on filters or solid culture medium. Alternatively, immature embryos or other target cells may be arranged on solid culture medium. The cells to be bombarded are positioned at an appropriate distance below the macroprojectile stopping plate.

3. Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986 and 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®.

Other examples of expression systems include STRATAGENE®'s COMPLETE CONTROL™ Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide.

It is contemplated that the proteins produced by the methods of the invention may be “overexpressed,” i.e., expressed in increased levels relative to its natural expression in cells. Such overexpression may be assessed by a variety of methods, including radio-labeling and/or protein purification. However, simple and direct methods are preferred, for example, those involving SDS/PAGE and protein staining or western blotting, followed by quantitative analyses, such as densitometric scanning of the resultant gel or blot. A specific increase in the level of the recombinant protein, polypeptide in comparison to the level in natural cells is indicative of overexpression, as is a relative abundance of the specific protein in relation to the other proteins produced by the host cell and, e.g., visible on a gel.

In some embodiments, the expressed proteinaceous sequence forms an inclusion body in the host cell, the host cells are lysed, for example, by disruption in a cell homogenizer, washed and/or centrifuged to separate the dense inclusion bodies and cell membranes from the soluble cell components. This centrifugation can be performed under conditions whereby the dense inclusion bodies are selectively enriched by incorporation of sugars, such as sucrose, into the buffer and centrifugation at a selective speed. Inclusion bodies may be solubilized in solutions containing high concentrations of urea (e.g., 8M) or chaotropic agents such as guanidine hydrochloride in the presence of reducing agents, such as β-mercaptoethanol or DTT (dithiothreitol), and refolded into a more desirable conformation, as would be known to one of ordinary skill in the art.

### 6. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Cell culture. HeLa-GFP cells were cultured in DMEM media supplemented with 10% heat-inactivated newborn calf serum as previously described (Montoya et al., 2004). Stock cultures were prewashed twice with PBS before the treatment of trypsin for cell resuspension and subsequently re-plated at the concentration of 4,000 cells/well. E. coli-GFP cells were grown in LB broth media while the MAC104 M. avium strain was grown in 7H9-ADC media.

HeLa-GFP fluorometric cytotoxicity assay. All cell lines were seeded in clear-bottomed 96-well assay plates (3603, Costar, Corning Inc., Corning, N.Y.) in order to minimized background fluorescence. HeLa cells were grown in a total volume of 0.2 ml of medium and incubated overnight for proper cell attachment. The quinone compounds were then added to each well (in triplicate) and then incubated for a period of 18 hr. For the determination of fluorescence, the assay plates were then read using the Fluoroskan Ascent F1 fluorometer (Thermo Electron Corp.) set at the excitation and emission parameters of 485 and 518 nm, respectively. Since the HeLa-GFP cell line is adherent, the fluorometer was set to read from the bottom of the plate for closer proximity to the cells. Before the readings were performed, all supernatant media was removed to avoid any background interference and thus enhancing the sensitivity of the optical readings. For calibration purposes, three untreated control wells were used to determine the maximum level of fluorescence (100%). It should be noted that 1 μl of DMSO was added to these control since this was the quinone solvent and previously shown to exhibit little if any cytotoxicity (Montoya et al., 2004). As a cytolysis control (complete loss of fluorescence), three wells containing cells were exposed to 0.2 ml of deionized water. These controls were then used to calculate the percent loss of GFP signal after compound exposure as determined by comparison with control wells containing cells that were not exposed to the chemical agent (see data analysis below).

Toxicity assays with GFP-expressing bacteria. E. coli was transformed with a GFP-expression plasmid that also confers tetracycline resistance was kindly provided by Dr. David Schneider (Elrod-Erickson et al., 2000). Mycobacterium avium strain MAC104 was transformed with p996A461 plasmid that confers hydromycin resistance and carries the GFP cassette under the control of the strong constitutive ribosomal promoter, rpsL, (for additional details see (Tran et al., 2004)). Both bacterial strains were grown and selected based on antibiotic resistance and GFP expression. All toxicity assays were performed using the plates and fluorometric parameters described in the previous section. Bacterial cell suspensions were seeded in a final volume of 100 μl at an optical density of 0.05 and 0.25 at 650 nm, for E. coli-GFP and M. avium-GFP, respectively. After exposure to the test compounds, assay plates were incubated at 37° C. in a humid environment to prevent dehydration in the wells. Unlike the HeLa-GFP assays, reading were performed without removing the supernatants to avoid loss of cells. Several untreated wells containing bacteria and media were used to determine the maximum level of fluorescence. Several wells containing media alone (no bacteria) were included in each assay to remove background fluorescence.

Data analysis. All compounds, including controls, were screened in triplicate. The average of the three wells that were used for the removal of the background fluorescence (wells containing water for the HeLa-GFP assays and wells containing media alone for bacteria) was subtracted from each individual well. The mean of these triplicate values for each compound was then obtained. These mean values were then divided by the average obtained for maximum fluorescence (cells not treated with compound) and then multiplied by 100 to obtain the percentage values as shown in Table 1. It should be noted that a few data points were derived from duplicate samples if the third reading was significantly different from the other two. Calculation of compound physical parameters, logP and MW, was performed with ChemOffice Ultra (Cambridge Soft) and this data is presented in the Supplementary materials section. Note that logP values were not calculated for two of the compounds, Q30-31. Correlation of physical properties with compound toxicity was performed via the Spearman Rank Correlation using SAS for Windows, version 7 (SAS Institute Inc.).

Fluorescent microscopy cytotoxicity assays. HeLa-GFP cells were plated on 24-well plates and left overnight to adhere. Different dilutions of the quinones were then added to each well (indicated in the legend) using the same concentrations of the positive and negative controls as described in the previous assay. Cell counts were done under fluorescent microscopy to determine cytotoxicity. The status of the fluorescent GFP-nuclei and the typical morphological characteristics of cells undergoing cell death were taken into account when differentiating between dying or viable cells as previously described (Montoya et al., 2004).

Compound synthesis. Naphthoquinone derivatives, Q2-Q44, were synthesized on solid support utilizing the Dotz reaction with solid supported Fischer carbene complexes. Plumbagin was purchased from Sigma. Test compounds were dissolved in DMSO at 20 mg/ml and stored at −20° C. until used.

### Example 2

### Results and Discussion

In order to rapidly screen novel compounds for their toxic properties, a relatively simple GFP-based assay was recently utilized to simultaneously screen several compounds without having to perform other elaborate assays (Montoya et al., 2004). Although easy to implement, this assay could not be applied to high-throughput analysis as it relied on the visualization of cell death (Montoya et al., 2004). Given this obvious limitation, the assay was modified to facilitate the simultaneous screening of multiple compounds in a 96-well format using an automated fluorescence plate reader. Since small quantities of compounds are required, this assay is particularly well suited for the screening of combinatorial chemical libraries. Another advantage of these microplate assays is the ability to perform all assays in duplicate or triplicate to derive more consistent results, and for this reason, all experiments were performed in triplicate.

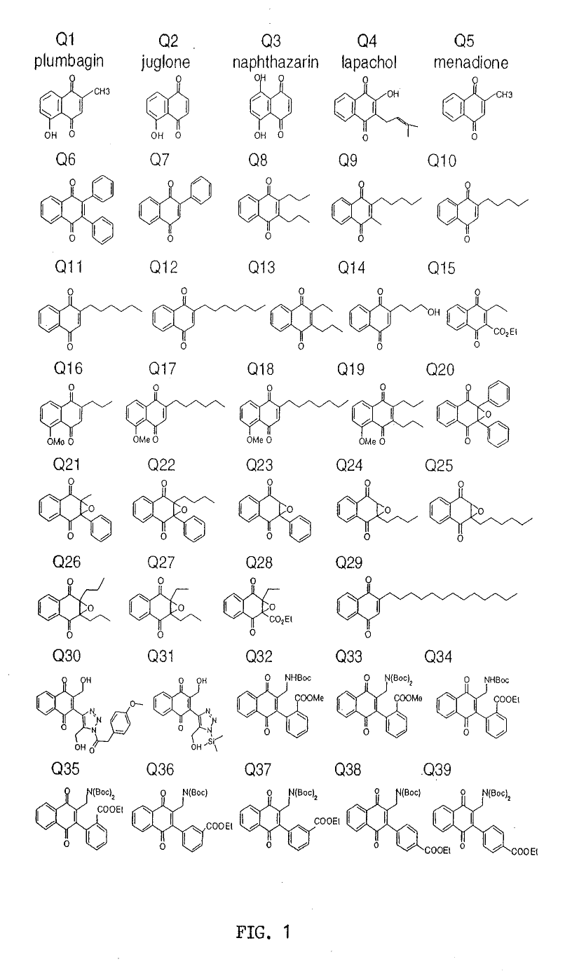

As proof of concept, the inventors tested thirty-nine naphthoquinone compounds (FIG. 1) on human HeLa-GFP cells in 96-well plates. As potentially important anti-microbial agents would not be detected with the HeLa-GFP assay alone, this approach was used to test the same compounds on GFP-expressing E. coli and M. avium strains. As can be seen in Table 1, the results show that both mammalian cells and bacteria can be analyzed in tandem to rapidly determine which compounds are specifically toxic to one of these cell types. It should be noted that this assay can only detect reduction of GFP signal (presumably due to cytolysis) but cannot differentiate the pathways or modes of death, which can subsequently be analyzed with well established assays. In these assays, plumbagin (Q1) was clearly the most toxic of the test compounds on both the mammalian cells and the two bacterial strains. In addition, two similar compounds, Q3 (naphthazarin) and Q5 (menadione; see FIG. 1) were also found to be highly toxic to all cell types. Apart from plumbagin, lapachol (Q4) was found to be the most toxic to M. avium as was previously determined with other assays (Tran et al., 2004). Interestingly, lapachol and similar quinones have recently been shown to exhibit strong inhibitory properties against two species of Leishmania associated to tegumentar leishmaniasis (Lima et al., 2004). Lapachol has also been used as an anticancer drug but found unsuitable for clinical use due to its toxic side effects (Block et al., 1974). This compound has recently been demonstrated to have anti-metastatic effects by inhibiting the invasiveness of cancer cells (Balassiano et al., 2005).

Although most of the compounds tested exhibited the highest level of toxicity at the higher concentration tested, several compounds were just as toxic at a ten-fold dilution (see Q1-3 ad Q23-25 at 0.2 μg, Table 1) against the HeLa-GFP cell line. Apart from differences in overall toxicity, two general types of toxic compounds were detected in these assays, those that exhibited toxicity to two or all three of the cell types (Q1-7) and those that were primarily toxic to the HeLa cells (Q24, Q26-28). As these two sets of compounds target different cell types, it is likely that these compounds will have different modes of action. Future experiments should help elucidate if these compounds have different molecular targets or if it is just a question of membrane permeability (see below). It is, however, important to point out that the observed differences would not have been detected if the compounds had been tested on a single cell type.

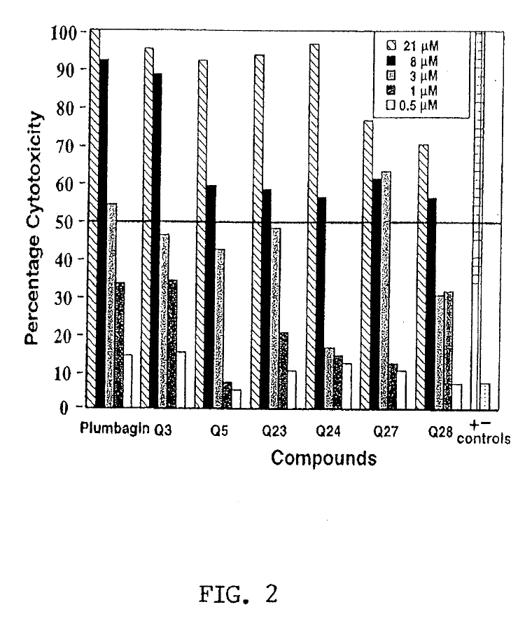

Several of the most toxic compounds were tested at various concentrations to determine the approximate LD50. As shown in FIG. 2, compounds Q1 and Q3 exhibited very similar LD50's of ˜3.0 μM while that of compounds Q2, Q5, Q23, and Q24 was between 3 to 8 μM. It is interesting to note that although compounds Q27 and Q28 were not as toxic as the other naphthoquinones at the highest concentrations, they had similar LD50 values as the other compounds (between 3 and 8 μM). In previous work, a subset of the naphthoquinone compounds (Q1-4 and Q8) were tested in a microbroth assay for bacteriocidal and bacteriostatic effects and found to have significant antimycobacterial activity against M. smegmatis, M. avium, and M. tuberculosis (Tran et al., 2004).

Activities against the three different types of cells and the molecular weight and compound solubility were analyzed for possible correlations. This analysis revealed that compound molecular weight significantly correlated with toxicity to HeLa cells (r=0.455, P=0.0019), with a weaker correlation to activity against M. avium (r=0.287, P=0.0592), and did not correlate with killing of E. coli (r=0.173, P=0.262). Solubility (quantified as logP), followed the same trend (HeLa r=0.622, P<0.0001, M. avium r=0.314, P=0.0586, E. coli r=0.222, P=0.1866). This would be expected, as much of the mass added to the larger compounds were alkyl or phenyl groups, which increase hydrophobicity. Interestingly, toxicity against the two prokaryotic organisms strongly and significantly correlated with each other (r=0.648, P<0.0001), but not with the eukaryotic cell line (E. coli to HeLa r=0.228, P=0.1361, M. avium to HeLa r=0.221, P=0.1488). This suggests that the mechanism of action of quinones is different against prokaryotic and eukaryotic cells, which has been observed in other studies as well (Tran et al., 2004). In addition, M. avium (mean viability after exposure 83.2±34.1) and E. coli (96.6±24.1) were more tolerant to the quinones than the HeLa cells (44.0±33.1). This is likely because of the great permeability barrier of mycobacteria (Nikaido, 2001) and the fact that E. coli often uses efflux pumps against quinones (Tegos et al., 2002). Compounds containing a 2,3-epoxide were highly toxic to eukaryotic cells (compounds Q20-Q28, mean viability post-exposure of HeLa 7.0±9.3), but exhibited no killing of prokaryotic cells (E. coli 102±22, M. avium 106±44). As mentioned earlier, the differences between the mammalian and prokaryotic cells could be attributed to the activation of different pathways and or modes of death.

Plumbagin, which was the most toxic compound to E. coli, is also the most toxic to M. avium, and is the third-most toxic to HeLa cells. Plumbagin and other similar quinones have been shown to mediate cell death by two distinct mechanisms involving redox cycling and reaction with reduced glutathione (Inbaraj and Chignell, 2004). Redox cycling results in formation of semiquinone radicals which then lead to the generation of superoxide anions and H₂0₂ (Inbaraj and Chignell, 2004). Furthermore, oxidative cycling induced by plumbagin appears to be the primary mechanism by which this compound kills mycobacteria (T. Primm, unpublished results). It is well known that reactive oxygen species (ROS) are involved in the cell death process and many apoptosis-inducing agents generate free radicals (Inbaraj and Chignell, 2004). In a recent report, plumbagin was demonstrated to induce apoptosis of human cervical cancer cells (ME-180 cell line) through the generation of ROS and a caspase-dependent pathway (Srinivas et al., 2004). Using cell-cytometry and known apoptotic markers, the inventors have shown that plumbagin and other naphthoqinones induce apoptosis in a lymphocyte cell line (Montoya et al., 2004). Interestingly, a significant level of necrosis was induced by the naphthoquinones and the positive control H₂0₂ (Montoya et al., 2004). Since oxidative stress has been shown to lead to necrosis (Samali et al., 1999; Cole and Perez-Polo, 2002); Bergamini et al., 2004), the detection of necrosis is consistent with the generation of ROS by these compounds as has been previously described (Inbaraj and Chignell, 2004; Srinivas et al., 2004). It is therefore very likely that both mechanisms are occurring simultaneously leading to the potent cytotoxic effects detected with the compounds that the inventors have tested thus far.

There are few effective antibiotics against Mycobacteria sp. and novel drugs are desperately needed against these pathogenic bacteria. A recent screen of acetophenone and naphthoquinone derivatives revealed that some of these compounds have significant anti-bacterial properties (Rajabi et al., 2005; Tran et al., 2004). Acetophenone-derivatives with the strongest antibiotic properties were subsequently tested in the HeLa-GFP cytotoxicity assay to determine if any of these compounds exhibited cytotoxic effects against mammalian cells (Rajabi et al., 2005). This analysis revealed that two of the most promising compounds were also highly toxic to mammalian cells. Several of the antibacterial acetophenone compounds exhibited mild cytotoxicity but most importantly three of these compounds were determined to exert their activity selectively against M. avium (Rajabi et al., 2005). Although current analysis of naphthoquinone derivatives on M. avium did not yield a compound with selective toxicity against this organism, it is anticipated that through extensive screening such a compound(s) will eventually be discovered. In addition to HeLa cells, the inventor has also created mammalian reporter cells using human fibroblasts (Hs 740) and human lung cancer (A549) cells, leading towards the ultimate goal of providing a panel of distinct cell lines that can be used for tandem screening assays.

TABLE 1
Percent-GFP-fluorescence after incubation with test compounds

| | HeLa-GFP^{a} | HeLa-GFP | E. coli-GFP^{c} | M. avium-GFP |
|---|---|---|---|---|
| Compound | (20 μg/ml)^{b} | (2 μg/ml) | (20 μg/ml) | (20 μg/ml) |
| Q1 | 9.0 | 13.0 | 1.0 | 7.0 |
| Q2 | 29.0 | 14.0 | 111.0 | 72.0 |
| Q3 | 4.0 | 0 | 41.0 | 38.0 |
| Q4 | 3.0 | 70.0 | 56.0 | 26.0 |
| Q5 | 22.0 | 13.0 | 58.0 | 69.0 |
| Q6 | 20.0 | 78.0 | 105.0 | 55.0 |
| Q7 | 25.0 | 13.0 | 89.0 | 35.0 |
| Q8 | 16.0 | 89.0 | 134.0 | 102.0 |
| Q9 | 31.0 | 90.0 | 115.0 | 85.0 |
| Q10 | 52.0 | 84.0 | 110.0 | 81.0 |
| Q11 | 14.0 | 84.0 | 98.0 | 69.0 |
| Q12 | 14.0 | 81.0 | 82.0 | 66.0 |
| Q13 | 21.0 | 89.0 | 93.0 | 72.0 |
| Q14 | 29.0 | 83.0 | 90.0 | 58.0 |
| Q15 | 53.0 | 85.0 | 80.0 | 40.0 |
| Q16 | 10.0 | 78.0 | 127.0 | 105.0 |
| Q17 | 18.0 | 80.0 | 115.0 | 100.0 |
| Q18 | 22.0 | 88.0 | 112.0 | 127.0 |
| Q19 | 38.0 | 78.0 | 99.0 | 73.0 |
| Q20 | 29.0 | 28.0 | 86.0 | 77.0 |
| Q21 | 23.0 | 57.0 | 122.0 | 57.0 |
| Q22 | 14.0 | 75.0 | 110.0 | 91.0 |
| Q23 | 3.0 | 14.0 | 56.0 | 66.0 |
| Q24 | 23.0 | 5.0 | 87.0 | 202.0 |
| Q25 | 18.0 | 0 | 118.0 | 133.0 |
| Q26 | 27.0 | 26.0 | 110.0 | 97.0 |
| Q27 | 21.0 | 21.0 | 117.0 | 133.0 |
| Q28 | 4.0 | 17.0 | 115.0 | 97.0 |
| Q29 | 19.0 | 80.0 | 100.0 | 105.0 |
| Q30 | 47.0 | 84.0 | 105.0 | 96.0 |
| Q31 | 82.0 | 88.0 | 107.0 | 94.0 |
| Q32 | 57.0 | 88.0 | 99.0 | 80.0 |
| Q33 | 62.0 | 88.0 | 102.0 | 74.0 |
| Q34 | 49.0 | 86.0 | 87.0 | 64.0 |
| Q35 | 58.0 | 85.0 | 104.0 | 108.0 |
| Q36 | 59.0 | 89.0 | 100.0 | 93.0 |
| Q37 | 62.0 | 90.0 | 105.0 | 92.0 |
| Q38 | 61.0 | 81.0 | 103.0 | 119.0 |
| Q39 | 62.0 | 88.0 | 108.0 | 96.0 |

^{a}HeLa and M. avium-GFP cells were incubated with compound for 18 h.
^{b}Compounds were tested at concentrations of 20 or 2 μg/ml as indicated. See Materials and methods for assay details.
^{c}E. coli-GFP was incubated with compound for 10 h.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods, and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the scope of the invention as defined by the appended claims.

### 7. REFERENCES

The following references, to the extent that they provide exemplary procedural or other details supplementary to those set forth herein, are specifically incorporated herein by reference.
U.S. Pat. No. 4,659,774
U.S. Pat. No. 4,683,202
U.S. Pat. No. 4,684,611
U.S. Pat. No. 4,816,571
U.S. Pat. No. 4,879,236
U.S. Pat. No. 4,952,500
U.S. Pat. No. 4,959,463
U.S. Pat. No. 5,141,813
U.S. Pat. No. 5,264,566
U.S. Pat. No. 5,302,523
U.S. Pat. No. 5,322,783
U.S. Pat. No. 5,384,253
U.S. Pat. No. 5,428,148
U.S. Pat. No. 5,464,765
U.S. Pat. No. 5,538,877
U.S. Pat. No. 5,538,880
U.S. Pat. No. 5,550,318
U.S. Pat. No. 5,554,744
U.S. Pat. No. 5,563,055
U.S. Pat. No. 5,574,146
U.S. Pat. No. 5,580,859
U.S. Pat. No. 5,589,466
U.S. Pat. No. 5,602,244
U.S. Pat. No. 5,610,042
U.S. Pat. No. 5,645,897
U.S. Pat. No. 5,656,610
U.S. Pat. No. 5,702,932
U.S. Pat. No. 5,705,629
U.S. Pat. No. 5,736,524
U.S. Pat. No. 5,780,448
U.S. Pat. No. 5,789,215
U.S. Pat. No. 5,871,986
U.S. Pat. No. 5,928,906
U.S. Pat. No. 5,945,100
U.S. Pat. No. 5,981,274
U.S. Pat. No. 5,994,624
U.S. Pat. No. 4,682,195
Ausubel et al., In: Current Protocols in Molecular Biology, John, Wiley & Sons, Inc, NY, 1996.
Balassiano et al., Oncol. Rep., 13:329-333, 2005.
Bates, Mol. Biotechnol., 2(2):135-145, 1994.
Battraw and Hall, Theor. App. Genet., 82(2):161-168, 1991.
Bergamini et al., Curr. Pharm. Des., 10:1611-1626, 2004.
Bhattacharjee et al., J. Plant Bioch. Biotech., 6(2):69-73. 1997.
Block et al., Cancer Chemother. Rep., 2(4):27-28, 1974.
Chen and Okayama, Mol. Cell Biol., 7(8):2745-2752, 1987.
Chiu et al., Electrophoresis, 23(3):449-455, 2002.
Christou et al., Proc. Natl. Acad. Sci. USA, 84(12):3962-3966, 1987.
Cole and Perez-Polo, J. Neurochem., 82:19-29, 2002.
D'Halluin et al., Plant Cell, 4(12):1495-1505, 1992.
Elrod-Erickson et al., Curr. Biol., 10:781-784, 2000.
EP 0 0273085
EP 0 266 032
Fechheimer, et al., Proc Natl. Acad. Sci. USA, 84:8463-8467, 1987.
Fraley et al., Proc. Natl. Acad. Sci. USA, 76:3348-3352, 1979.
Frankfurt and Krishan, Anticancer Drugs, 14:555-561, 2003a.
Frankfurt et al., J. Biomol. Screen, 8:185-190, 2003b.
Froehler et al., Nucleic Acids Res., 14(13):5399-5407, 1986.
Gopal, Mol. Cell Biol., 5:1188-1190, 1985.
Graham and Van Der Eb, Virology, 52:456-467, 1973.
Harland and Weintraub, J. Cell Biol., 101(3):1094-1099, 1985.
He et al., 1994
Heim et al., Proc. Natl. Acad. Sci. USA, 91(26):12501-12504, 1994.
Hong et al., Plant Mol. Biol., 34(3):549-555, 1997.
Hou and Lin, Plant Physiology, 111: 166, 1996.
Inbaraj and Chignell, Chem. Res. Toxicol., 17:55-62, 2004.
Inouye and Inouye, Nucleic Acids Res., 13: 3101-3109, 1985.
Kaeppler et al., Plant Cell Rep., 8:415-418, 1990.
Kanda et al., Curr. Biol., 8:377-385, 1998.
Kaneda et al., Science, 243:375-378, 1989.
Karasawa et al., Biochem. J., 381(Pt 1):307-312, 2004.
Kato et al, J. Biol. Chem., 266:3361-3364, 1991.
Kenseth and Coldiron, Curr. Opin. Chem. Biol., 8:418-423, 2004.
Klein et al., Nature, 327:70-73, 1987.
Lazzeri, Methods Mol. Biol., 49:95-106, 1995.
Lee et al., Environ. Mol. Mutagen., 13(1):54-59, 1989.
Lima et al., Mem. Inst. Oswaldo Cruz, 99:757-761, 2004.
Maniatis, et al., Molecular Cloning, A Laboratory Manual, Cold Spring Harbor Press, Cold Spring Harbor, N.Y., 1990.
Montoya et al., Biochem. Biophys. Res. Commun., 325:1517-1523, 2004.
Nabel et al., Science, 244(4910):1342-1344, 1989.
Nicolau and Sene, Biochim. Biophys. Acta, 721:185-190, 1982.
Nicolau et al., Methods Enzymol., 149:157-176, 1987.
Nikaido, Semin. Cell Dev. Biol., 12:215-223, 2001.
Omirulleh et al., Plant Mol. Biol., 21(3):415-428, 1993.
PCT Appln. WO 9217598
PCT Appln. WO 94/09699
PCT Appln. WO 94/09699
PCT Appln. WO 95/06128
Perales et al., Proc. Natl. Acad. Sci. USA, 91:4086-4090, 1994.
Potrykus et al., Mol. Gen. Genet., 199(2):169-177, 1985.
Potrykus et al., Mol. Gen. Genet., 199:183-188, 1985.
Potter et al., Proc. Natl. Acad. Sci. USA, 81:7161-7165, 1984.
Prasher et al., Gene, 111(2):229-233, 1992.
Rajabi et al., Lett. Appl. Microbiol., 40:212-217, 2005.
Reddy et al., Gene Ther., 9(22):1542-1550, 2002.
Rhodes et al., Methods Mol. Biol., 55:121-131, 1995.
Rippe, et al., Mol. Cell Biol., 10:689-695, 1990.
Samali et al., Biochem. Biophys. Res. Commun., 255:6-11, 1999.
Sambrook and Russell, Molecular Cloning: A Laboratory Manual 3^{rd} Ed., Cold Spring Harbor Laboratory Press, 2001.
Srinivas et al., Mol. Carcinog., 40:201-211, 2004.
Tegos et al., Antimicrob. Agents Chemother., 46:3133-3141, 2002.
Tran et al., Bioorg. Med. Chem., 12:4809-4813, 2004.
Tsien, Annu. Rev. Biochem., 67:509-544, 1998.
Tsukada et al., Plant Cell Physiol., 30(4)599-604, 1989.
Tur-Kaspa et al., Mol. Cell Biol., 6:716-718, 1986.
Wagner et al., Proc. Natl. Acad. Sci. USA 87(9):3410-3414, 1990.
Wilson et al., Science, 244:1344-1346, 1989.
Wong et al., Gene, 10:87-94, 1980.
Wu and Wu, Adv. Drug Delivery Rev., 12:159-167, 1993.
Wu and Wu, Biochemistry, 27: 887-892, 1988.
Wu and Wu, J. Biol. Chem., 262:4429-4432, 1987.
Yang and Russell, Proc. Natl. Acad. Sci. USA, 87:4144-4148, 1990.
Zemanova et al., Drug Discov. Today, 8:1085-1093, 2003.
Zhou et al., Nature, 361(6412):543-547, 1993. Kassel, Curr. Opin. Chem. Biol., 8:339-345, 2004.

What is claimed is:

1. A method of screening a test substance for cytotoxic and antimicrobial activity comprising:
(a) providing at least one eukaryotic cell and at least one pathogenic organism, each of said cells expressing a fluorescent marker protein or stained with a fluorescent dye;
(b) contacting each of said cells with said test substance; and
(c) assessing a fluorescent signal from each of said cell and organism,
wherein a decrease in signal from only said eukaryotic cell indicates that the test substance is a cytotoxic agent, and wherein a decrease in signal from only said pathogenic organism indicates that the test substance is an antimicrobial agent, and wherein a decrease in signal from both said eukaryotic cell and said pathogenic organism indicates that the test substance is both a cytotoxic agent and an antimicrobial agent.

2. The method of claim 1, wherein said eukaryotic cell is a cancer cell.

3. The method of claim 2, wherein said cancer cell is a multi-drug resistant cancer cell.

4. The method of claim 1, wherein said pathogenic organism is a bacterium or protozoan.

5. The method of claim 4, wherein said bacterial cell is a multi-drug resistant bacterial cell.

6. The method of claim 1, wherein said fluorescent marker protein is GFP, BFP, CFP, YFP, EGFP, EYFP, Venus, Citrine, phiYFP, copGFP CGFP, ECFP, Cerulean, CyPet, T-Sapphire, Emerald, YPet, AcGFP1, AmCyan, AsRed2, dsRed, dsRed2, dsRed-Express, EBFP, HcRed, ZsGreen, ZsYellow, J-Red, TurboGFP, Kusabira Orange, Midoriishi Cyan, mOrange, DsRed-monomer, mStrawberry, mRFP1, tdTomato, mCherry, mPlum, and mRaspberry.

7. The method of claim 1, wherein said fluorescent dye is CellTracker Green™.

8. The method of claim 1, wherein providing comprises distributing said eukaryotic cell into at least one well of a first multi-well plate, and distributing said pathogenic organism into at least one well of a second multi-well plate.

9. The method of claim 8, wherein said multi-well plate is a 6-well, 24-well, 96-well or high throughput plate.

10. The method of claim 1, wherein assessing comprises fluorescence detection via commercially available devices.

11. The method of claim 1, further comprising assessing the fluorescent signal from a control eukaryotic cell and control pathogenic organism that each express the same fluorescent marker protein as the cells in step (a), wherein said controls have not been treated with said test substance.

12. The method of claim 1, where the fluorescent marker protein or dye in said eukaryotic cell and pathogenic organism is the same

13. The method of claim 1, where the fluorescent marker protein or dye in said eukaryotic cell and pathogenic organism is different.

14. The method of claim 1, wherein the test substance is a protein, a peptide, a nucleic acid, an organopharmaceutical, a lipid, or a carbohydrate.

15. The method of claim 1, further comprising more than one concentration of said test substance against each of said cells.

16. The method of claim 15, further comprising testing multiple replicates of each cell and concentration tested.
